(19) United States
(12) Patent Application Publication
(10) Pub. No.: US 2026/0284135 A1
(43) Pub. Date: Sep. 24, 2026

(54) METHOD FOR PREPARING AROMATIC ESSENTIAL OIL FOR CLEARING NASAL PASSAGES

(71) Applicant: Zhuhai Hengqin Gende Biotechnology Co., Ltd., Zhuhai (CN)

(72) Inventor: Jianhua Lai, Zhuhai (CN)

(21) Appl. No.: 19/201,085

(22) Filed: May 7, 2025

(30) Foreign Application Priority Data
Mar. 19, 2025 (CN) 202510325431.3

(51) Int. Cl. A61K 36/61; A61K 9/00; A61K 31/045; A61K 31/125; A61K 36/185; A61K 36/534; A61K 36/87; A61P 11/02

(52) U.S. Cl. CPC A61K 36/61; A61K 9/0043; A61K 31/045; A61K 31/125; A61K 36/185; A61K 36/534; A61K 36/87; A61P 11/02; A61K 2236/15; A61K 2236/331; A61K 2236/35

(57) ABSTRACT

A method for preparing aromatic essential oil for clearing nasal passages, including the following steps: using grape seeds, jojoba seeds, Melaleuca alternifolia leaves, Melaleuca viridiflora leaves, Eucalyptus globulus leaves, mint, camphor, and borneol as raw materials, distilling to obtain grape seed oil, jojoba seed oil, Melaleuca alternifolia leaf oil, Melaleuca viridiflora leaf oil, Eucalyptus globulus leaf oil, mint essential oil, camphor oil, and borneol oil, and then mixing the grape seed oil, jojoba seed oil, Melaleuca alternifolia leaf oil, Melaleuca viridiflora leaf oil, Eucalyptus globulus leaf oil, mint essential oil, camphor oil, and borneol oil to obtain the aromatic essential oil for clearing nasal passages. Compared with the preparation of the aromatic essential oil using commercially available raw materials, the aromatic essential oil prepared by the distillation method provided by the invention has a better rhinitis treatment effect.

### TECHNICAL FIELD

The invention relates to the technical field of cosmetics, and in particular to a method for preparing aromatic essential oil for clearing nasal passages.

### BACKGROUND ART

Rhinitis is an inflammatory disease of the nasal mucosa and submucosal tissue. The causes are relatively complex, mainly including viral infection, bacterial infection, contact with allergens and inhalation of irritating gases; the symptoms of rhinitis mainly include nasal congestion, runny nose, sneezing, headache and dizziness, etc. These symptoms will affect the patient's daily life and work to a certain extent.

Aromatic essential oil is a plant-based skincare product formulated from natural botanicals. Enriched with aromatic compounds, it embodies the traditional Chinese medicinal properties of “opening bodily orifices through fragrance and directing therapeutic effects upward”. Studies have shown that the aromatic ingredients in aromatic essential oil can repair and maintain the nasal mucosa and promote the regeneration of nasal neurons. Therefore, aromatic essential oil has a certain therapeutic potential for rhinitis.

However, some existing aromatic essential oils are not ideal for the treatment of rhinitis. Therefore, developing an aromatic essential oil with better therapeutic effect on rhinitis is expected to further expand the application of aromatic essential oils in the treatment of rhinitis.

### SUMMARY OF THE INVENTION

The purpose of the invention is to provide a method for preparing aromatic essential oil for clearing nasal passages. The fragrant essential oil for clearing nasal passages prepared by the invention has a good therapeutic effect on rhinitis.

In order to achieve the above purpose, the invention provides the following technical solutions.

A method for preparing aromatic essential oil for clearing nasal passages, including the following steps:
using grape seeds, jojoba seeds, Melaleuca alternifolia leaves, Melaleuca viridiflora leaves, Eucalyptus globulus leaves, mint, camphor, and borneol as raw materials, distilling to obtain grape seed oil, jojoba seed oil, Melaleuca alternifolia leaf oil, Melaleuca viridiflora leaf oil, Eucalyptus globulus leaf oil, mint essential oil, camphor oil, and borneol oil, and then mixing the grape seed oil, jojoba seed oil, Melaleuca alternifolia leaf oil, Melaleuca viridiflora leaf oil, Eucalyptus globulus leaf oil, mint essential oil, camphor oil, and borneol oil to obtain the aromatic essential oil for clearing nasal passages.

Further, the method for preparing the grape seed oil includes the following steps:
grinding grape seeds into a powder through a 60-mesh sieve to obtain grape seed powder, mixing the grape seed powder with water at a solid-to-liquid ratio of 1:(5-10), and distilling at 120° C. for 4-6 hours to obtain the grape seed oil.

Further, the method for preparing the jojoba seed oil includes the following steps:
grinding jojoba seeds into a powder through a 60-mesh sieve to obtain jojoba seed powder, mixing the jojoba seed powder with water at a solid-to-liquid ratio of 1:(5-10), and distilling at 140° C. for 2-3 hours to obtain the jojoba seed oil.

Further, the method for preparing the Melaleuca alternifolia leaf oil includes the following steps:
grinding Melaleuca alternifolia leaves into a powder through a 60-mesh sieve to obtain Melaleuca alternifolia leaf powder, mixing the Melaleuca alternifolia leaf powder with hexane-water (volume ratio 3:2) at a solid-to-liquid ratio of 1:(10-15), and distilling at 80° C. for 4-6 hours to obtain the Melaleuca alternifolia leaf oil.

Further, the method for preparing the Melaleuca viridiflora leaf oil includes the following steps:
grinding Melaleuca viridiflora leaves into a powder through a 60-mesh sieve to obtain Melaleuca viridiflora leaf powder, mixing the Melaleuca viridiflora leaf powder with hexane-water (volume ratio 3:2) at a solid-to-liquid ratio of 1:(10-15), and distilling at 95° C. for 3-5 hours to obtain the Melaleuca viridiflora leaf oil.

Further, the method for preparing the Eucalyptus globulus leaf oil includes the following steps:
grinding Eucalyptus globulus leaves into a powder through a 60-mesh sieve to obtain Eucalyptus globulus leaf powder, mixing the Eucalyptus globulus leaf powder with ether-water (volume ratio 1:3) at a solid-to-liquid ratio of 1:(4-8), and distilling at 60° C. for 1-3 hours to obtain the Eucalyptus globulus leaf oil.

Further, the method for preparing the mint essential oil includes the following steps:
grinding mint into a powder through a 60-mesh sieve to obtain mint powder, mixing the mint powder with water at a solid-to-liquid ratio of 1:(5-10), and distilling at 100° C. for 0.5-1.5 hours to obtain the mint essential oil.

Further, the method for preparing the camphor oil includes the following steps:
grinding camphor into a powder through a 60-mesh sieve to obtain camphor powder, mixing the camphor powder with water at a solid-to-liquid ratio of 1:(5-10), and distilling at 120° C. for 1-2 hours to obtain the mint camphor oil.

Further, the method for preparing the borneol oil includes the following steps:
grinding borneol into a powder through a 60-mesh sieve to obtain borneol powder, mixing the borneol powder with ether-water (volume ratio 1:3) at a solid-to-liquid ratio of 1:(4-8), and distilling at 50° C. for 2-3 hours to obtain the borneol oil.

Compared with the prior art, the invention has the following beneficial effects.

The aromatic essential oil for clearing nasal passages provided by the invention can quickly relieve nasal congestion within two minutes. Compared with the preparation of the aromatic essential oil using commercially available raw materials, the aromatic essential oil prepared by the distillation method provided by the invention has a better effect of quickly relieving nasal congestion.

The aromatic essential oil for clearing nasal passages provided by the invention can effectively treat rhinitis. Compared with the preparation of the aromatic essential oil using commercially available raw materials, the aromatic essential oil prepared by the distillation method provided by the invention has a better rhinitis treatment effect.

### SPECIFIC EMBODIMENTS OF THE INVENTION

Now, various exemplary embodiments of the invention are described in detail, and this detailed description should not be considered as a limitation of the invention, but should be understood as a more detailed description of certain aspects, characteristics and embodiments of the invention. It should be understood that the terms described in the invention are only for describing specific embodiments and are not used to limit the invention.

In addition, for the numerical range in the invention, it is understood that each intermediate value between the upper and lower limits of the range is also specifically disclosed. Each smaller range between the intermediate value in any stated value or stated range and any other stated value or intermediate value in the range is also included in the invention. The upper and lower limits of these smaller ranges can be independently included or excluded in the scope.

Unless otherwise indicated, all technical and scientific terms used herein have the same meanings as those generally understood by those skilled in the art. Although the invention describes only preferred methods and materials, any methods and materials similar or equivalent to those described herein may also be used in the implementation or testing of the invention. All documents mentioned in this specification are incorporated by reference to disclose and describe the methods and/or materials associated with the documents. In the event of a conflict with any incorporated document, the content of this specification shall prevail.

It will be apparent to those skilled in the art that various modifications and variations may be made to the specific embodiments of the invention description without departing from the scope or spirit of the invention. Other embodiments derived from the invention description will be apparent to the those skilled in the art. The invention description and examples are exemplary only.

The words “include,” “comprise,” “have,” “contain,” etc. used in this document are open-ended terms, meaning including but not limited to.

In the following embodiments, a method for preparing aromatic essential oil for clearing nasal passages, including the following steps:
1) grinding grape seeds into a powder through a 60-mesh sieve to obtain grape seed powder, mixing the grape seed powder with water at a solid-to-liquid ratio of 1:(5-10), and distilling at 120° C. for 4-6 hours to obtain the grape seed oil;
2) grinding jojoba seeds into a powder through a 60-mesh sieve to obtain jojoba seed powder, mixing the jojoba seed powder with water at a solid-to-liquid ratio of 1:(5-10), and distilling at 140° C. for 2-3 hours to obtain the jojoba seed oil;
3) grinding Melaleuca alternifolia leaves into a powder through a 60-mesh sieve to obtain Melaleuca alternifolia leaf powder, mixing the Melaleuca alternifolia leaf powder with hexane-water (volume ratio 3:2) at a solid-to-liquid ratio of 1:(10-15), and distilling at 80° C. for 4-6 hours to obtain the Melaleuca alternifolia leaf oil;
4) grinding Melaleuca viridiflora leaves into a powder through a 60-mesh sieve to obtain Melaleuca viridiflora leaf powder, mixing the Melaleuca viridiflora leaf powder with hexane-water (volume ratio 3:2) at a solid-to-liquid ratio of 1:(10-15), and distilling at 95° C. for 3-5 hours to obtain the Melaleuca viridiflora leaf oil;
5) grinding Eucalyptus globulus leaves into a powder through a 60-mesh sieve to obtain Eucalyptus globulus leaf powder, mixing the Eucalyptus globulus leaf powder with ether-water (volume ratio 1:3) at a solid-to-liquid ratio of 1:(4-8), and distilling at 60° C. for 1-3 hours to obtain the Eucalyptus globulus leaf oil;
6) grinding mint into a powder through a 60-mesh sieve to obtain mint powder, mixing the mint powder with water at a solid-to-liquid ratio of 1:(5-10), and distilling at 100° C. for 0.5-1.5 hours to obtain the mint essential oil;
7) grinding camphor into a powder through a 60-mesh sieve to obtain camphor powder, mixing the camphor powder with water at a solid-to-liquid ratio of 1:(5-10), and distilling at 120° C. for 1-2 hours to obtain the mint camphor oil;
8) grinding borneol into a powder through a 60-mesh sieve to obtain borneol powder, mixing the borneol powder with ether-water (volume ratio 1:3) at a solid-to-liquid ratio of 1:(4-8), and distilling at 50° C. for 2-3 hours to obtain the borneol oil;
9) weighing each raw material according to the following mass parts:
40 parts of grape seed oil, 33 parts of jojoba seed oil, 3 parts of Melaleuca alternifolia leaf oil, 2 parts of Melaleuca viridiflora leaf oil, 2 parts of Eucalyptus globulus leaf oil, 5 parts of mint essential oil, 2 parts of camphor oil, 1 part of borneol oil, and 1 part of tocopherol acetate;
10) weighing the raw materials weighed in step 1), stirring at room temperature for 40 minutes, then allowing to stand at room temperature for 30 minutes, and finally stirring at room temperature for 20 minutes to obtain the aromatic essential oil for clearing nasal passages.

### Embodiment 1

A Method for Preparing Aromatic Essential Oil for Clearing Nasal Passages
1) grinding grape seeds into a powder through a 60-mesh sieve to obtain grape seed powder, mixing the grape seed powder with water at a solid-to-liquid ratio of 1:5, and distilling at 120° C. for 4 hours to obtain the grape seed oil;
2) grinding jojoba seeds into a powder through a 60-mesh sieve to obtain jojoba seed powder, mixing the jojoba seed powder with water at a solid-to-liquid ratio of 1:5, and distilling at 140° C. for 2 hours to obtain the jojoba seed oil;
3) grinding Melaleuca alternifolia leaves into a powder through a 60-mesh sieve to obtain Melaleuca alternifolia leaf powder, mixing the Melaleuca alternifolia leaf powder with hexane-water (volume ratio 3:2) at a solid-to-liquid ratio of 1:10, and distilling at 80° C. for 4 hours to obtain the Melaleuca alternifolia leaf oil;
4) grinding Melaleuca viridiflora leaves into a powder through a 60-mesh sieve to obtain Melaleuca viridiflora leaf powder, mixing the Melaleuca viridiflora leaf powder with hexane-water (volume ratio 3:2) at a solid-to-liquid ratio of 1:10, and distilling at 95° C. for 3 hours to obtain the Melaleuca viridiflora leaf oil;
5) grinding Eucalyptus globulus leaves into a powder through a 60-mesh sieve to obtain Eucalyptus globulus leaf powder, mixing the Eucalyptus globulus leaf powder with ether-water (volume ratio 1:3) at a solid-to-liquid ratio of 1:4, and distilling at 60° C. for 1 hour to obtain the Eucalyptus globulus leaf oil;
6) grinding mint into a powder through a 60-mesh sieve to obtain mint powder, mixing the mint powder with water at a solid-to-liquid ratio of 1:5, and distilling at 100° C. for 0.5 hour to obtain the mint essential oil;
7) grinding camphor into a powder through a 60-mesh sieve to obtain camphor powder, mixing the camphor powder with water at a solid-to-liquid ratio of 1:5, and distilling at 120° C. for 1 hour to obtain the mint camphor oil;
8) grinding borneol into a powder through a 60-mesh sieve to obtain borneol powder, mixing the borneol powder with ether-water (volume ratio 1:3) at a solid-to-liquid ratio of 1:4, and distilling at 50° C. for 2 hours to obtain the borneol oil;
9) weighing each raw material according to the following mass parts:
40 parts of grape seed oil, 33 parts of jojoba seed oil, 3 parts of Melaleuca alternifolia leaf oil, 2 parts of Melaleuca viridiflora leaf oil, 2 parts of Eucalyptus globulus leaf oil, 5 parts of mint essential oil, 2 parts of camphor oil, 1 part of borneol oil, and 1 part of tocopherol acetate;
10) weighing the raw materials weighed in step 1), stirring at room temperature for 40 minutes, then allowing to stand at room temperature for 30 minutes, and finally stirring at room temperature for 20 minutes to obtain the aromatic essential oil for clearing nasal passages.

### Embodiment 2

A Method for Preparing Aromatic Essential Oil for Clearing Nasal Passages
1) grinding grape seeds into a powder through a 60-mesh sieve to obtain grape seed powder, mixing the grape seed powder with water at a solid-to-liquid ratio of 1:8, and distilling at 120° C. for 5 hours to obtain the grape seed oil;
2) grinding jojoba seeds into a powder through a 60-mesh sieve to obtain jojoba seed powder, mixing the jojoba seed powder with water at a solid-to-liquid ratio of 1:8, and distilling at 140° C. for 2.5 hours to obtain the jojoba seed oil;
3) grinding Melaleuca alternifolia leaves into a powder through a 60-mesh sieve to obtain Melaleuca alternifolia leaf powder, mixing the Melaleuca alternifolia leaf powder with hexane-water (volume ratio 3:2) at a solid-to-liquid ratio of 1:12, and distilling at 80° C. for 5 hours to obtain the Melaleuca alternifolia leaf oil;
4) grinding Melaleuca viridiflora leaves into a powder through a 60-mesh sieve to obtain Melaleuca viridiflora leaf powder, mixing the Melaleuca viridiflora leaf powder with hexane-water (volume ratio 3:2) at a solid-to-liquid ratio of 1:12, and distilling at 95° C. for 4 hours to obtain the Melaleuca viridiflora leaf oil;
5) grinding Eucalyptus globulus leaves into a powder through a 60-mesh sieve to obtain Eucalyptus globulus leaf powder, mixing the Eucalyptus globulus leaf powder with ether-water (volume ratio 1:3) at a solid-to-liquid ratio of 1:6, and distilling at 60° C. for 2 hours to obtain the Eucalyptus globulus leaf oil;
6) grinding mint into a powder through a 60-mesh sieve to obtain mint powder, mixing the mint powder with water at a solid-to-liquid ratio of 1:8, and distilling at 100° C. for 1 hour to obtain the mint essential oil;
7) grinding camphor into a powder through a 60-mesh sieve to obtain camphor powder, mixing the camphor powder with water at a solid-to-liquid ratio of 1:8, and distilling at 120° C. for 1.5 hours to obtain the mint camphor oil;
8) grinding borneol into a powder through a 60-mesh sieve to obtain borneol powder, mixing the borneol powder with ether-water (volume ratio 1:3) at a solid-to-liquid ratio of 1:6, and distilling at 50° C. for 2.5 hours to obtain the borneol oil;
9) weighing each raw material according to the following mass parts:
40 parts of grape seed oil, 33 parts of jojoba seed oil, 3 parts of Melaleuca alternifolia leaf oil, 2 parts of Melaleuca viridiflora leaf oil, 2 parts of Eucalyptus globulus leaf oil, 5 parts of mint essential oil, 2 parts of camphor oil, 1 part of borneol oil, and 1 part of tocopherol acetate;
10) weighing the raw materials weighed in step 1), stirring at room temperature for 40 minutes, then allowing to stand at room temperature for 30 minutes, and finally stirring at room temperature for 20 minutes to obtain the aromatic essential oil for clearing nasal passages.

### Embodiment 3

A Method for Preparing Aromatic Essential Oil for Clearing Nasal Passages
1) grinding grape seeds into a powder through a 60-mesh sieve to obtain grape seed powder, mixing the grape seed powder with water at a solid-to-liquid ratio of 1:10, and distilling at 120° C. for 6 hours to obtain the grape seed oil;
2) grinding jojoba seeds into a powder through a 60-mesh sieve to obtain jojoba seed powder, mixing the jojoba seed powder with water at a solid-to-liquid ratio of 1:10, and distilling at 140° C. for 3 hours to obtain the jojoba seed oil;
3) grinding Melaleuca alternifolia leaves into a powder through a 60-mesh sieve to obtain Melaleuca alternifolia leaf powder, mixing the Melaleuca alternifolia leaf powder with hexane-water (volume ratio 3:2) at a solid-to-liquid ratio of 1:15, and distilling at 80° C. for 6 hours to obtain the Melaleuca alternifolia leaf oil;
4) grinding Melaleuca viridiflora leaves into a powder through a 60-mesh sieve to obtain Melaleuca viridiflora leaf powder, mixing the Melaleuca viridiflora leaf powder with hexane-water (volume ratio 3:2) at a solid-to-liquid ratio of 1:15, and distilling at 95° C. for 5 hours to obtain the Melaleuca viridiflora leaf oil;
5) grinding Eucalyptus globulus leaves into a powder through a 60-mesh sieve to obtain Eucalyptus globulus leaf powder, mixing the Eucalyptus globulus leaf powder with ether-water (volume ratio 1:3) at a solid-to-liquid ratio of 1:8, and distilling at 60° C. for 3 hours to obtain the Eucalyptus globulus leaf oil;
6) grinding mint into a powder through a 60-mesh sieve to obtain mint powder, mixing the mint powder with water at a solid-to-liquid ratio of 1:10, and distilling at 100° C. for 1.5 hours to obtain the mint essential oil;
7) grinding camphor into a powder through a 60-mesh sieve to obtain camphor powder, mixing the camphor powder with water at a solid-to-liquid ratio of 1:10, and distilling at 120° C. for 2 hours to obtain the mint camphor oil;
8) grinding borneol into a powder through a 60-mesh sieve to obtain borneol powder, mixing the borneol powder with ether-water (volume ratio 1:3) at a solid-to-liquid ratio of 1:8, and distilling at 50° C. for 3 hours to obtain the borneol oil;
9) weighing each raw material according to the following mass parts:
40 parts of grape seed oil, 33 parts of jojoba seed oil, 3 parts of Melaleuca alternifolia leaf oil, 2 parts of Melaleuca viridiflora leaf oil, 2 parts of Eucalyptus globulus leaf oil, 5 parts of mint essential oil, 2 parts of camphor oil, 1 part of borneol oil, and 1 part of tocopherol acetate;
10) weighing the raw materials weighed in step 1), stirring at room temperature for 40 minutes, then allowing to stand at room temperature for 30 minutes, and finally stirring at room temperature for 20 minutes to obtain the aromatic essential oil for clearing nasal passages.

### Comparative Embodiment 1

### A Method for Preparing Aromatic Essential Oil for Clearing Nasal Passages

It is the same as Embodiment 2, except that step 3) is:
grinding Melaleuca alternifolia leaves into a powder through a 60-mesh sieve to obtain Melaleuca alternifolia leaf powder, mixing the Melaleuca alternifolia leaf powder with water at a solid-to-liquid ratio of 1:12, and distilling at 80° C. for 5 hours to obtain the Melaleuca alternifolia leaf oil.

### Comparative Embodiment 2

### A Method for Preparing Aromatic Essential Oil for Clearing Nasal Passages

It is the same as Embodiment 2, except that step 4) is:
grinding Melaleuca viridiflora leaves into a powder through a 60-mesh sieve to obtain Melaleuca viridiflora leaf powder, mixing the Melaleuca viridiflora leaf powder with water at a solid-to-liquid ratio of 1:12, and distilling at 95° C. for 4 hours to obtain the Melaleuca viridiflora leaf oil.

### Comparative Embodiment 3

### A Method for Preparing Aromatic Essential Oil for Clearing Nasal Passages

It is the same as Embodiment 2, except that step 5) is:
grinding Eucalyptus globulus leaves into a powder through a 60-mesh sieve to obtain Eucalyptus globulus leaf powder, mixing the Eucalyptus globulus leaf powder with water at a solid-to-liquid ratio of 1:6, and distilling at 60° C. for 2 hours to obtain the Eucalyptus globulus leaf oil.

### Comparative Embodiment 4

### A Method for Preparing Aromatic Essential Oil for Clearing Nasal Passages

It is the same as Embodiment 2, except that step 8) is:
grinding borneol into a powder through a 60-mesh sieve to obtain borneol powder, mixing the borneol powder with water at a solid-to-liquid ratio of 1:6, and distilling at 50° C. for 2.5 hours to obtain the borneol oil.

### Comparative Embodiment 5

### A Method for Preparing Aromatic Essential Oil for Clearing Nasal Passages

Using commercially available grape seed oil, jojoba seed oil, Melaleuca alternifolia leaf oil, Melaleuca viridiflora leaf oil, Eucalyptus globulus leaf oil, mint essential oil, camphor oil, and borneol oil to prepare aromatic essential oil for clearing nasal passages.

### Effect Verification

### 1. Effect Verification of Aromatic Essential Oils for Clearing Nasal Passages in Quickly Relieving Nasal Congestion

160 patients with rhinitis, aged 30 to 40 years old, with a course of disease of 2 to 3 years, were selected and randomly divided into 8 groups, 20 cases in each group, and the aromatic essential oils prepared in Embodiments 1 to 3 and Comparative Embodiments 1 to 5 were applied respectively. The specific applying method was: when the patient felt nasal congestion, the aromatic essential oil was applied to the Yingxiang (LI20), Renzhong (GV26), and Suliao (GV25) acupoints, and applied gently and evenly.

The validation results of the effectiveness of aromatic essential oils in rapidly relieving nasal congestion are shown in Table 1.

TABLE 1
Verification Results of the Effect of Aromatic Essential
Oils in Quickly Relieving Nasal Congestion

| | Relief Within | Relief Within | Relief Within |
|---|---|---|---|
| | 2 Minutes | 3 Minutes | 5 Minutes |
| Embodiment | √ | | |
| 1 | | | |
| Embodiment | √ | | |
| 2 | | | |
| Embodiment | √ | | |
| 3 | | | |
| Comparative | | | √ |
| Embodiment | | | |
| 1 | | | |
| Comparative | | | √ |
| Embodiment | | | |
| 2 | | | |
| Comparative | | √ | |
| Embodiment | | | |
| 3 | | | |
| Comparative | | | √ |
| Embodiment | | | |
| 4 | | | |
| Comparative | | √ | |
| Embodiment | | | |
| 5 | | | |

It can be seen from the data in Table 1 that the aromatic essential oil for clearing nasal passages provided by the invention can quickly relieve nasal congestion within 2 minutes. Compared with the preparation of aromatic essential oil using commercially available raw materials, the aromatic essential oil prepared by the distillation method provided by the invention has a better effect of quickly relieving nasal congestion; in addition, changing the distillation method of each aromatic essential oil will lead to a decrease in the effect of quickly relieving nasal congestion to varying degrees.

### 2. Therapeutic Effect of Aromatic Essential Oil on Rhinitis

400 patients with rhinitis, aged 30 to 40 years, with a course of disease of 2 to 3 years, were selected and randomly divided into 8 groups, 50 cases in each group, and the aromatic essential oils prepared in Embodiments 1 to 3 and Comparative Embodiments 1 to 5 were applied respectively, and the effective rate was statistically analyzed after 14 days; the specific applying method was: applying the aromatic essential oil to the Yingxiang (LI20), Renzhong (GV26), and Suliao (GV25) acupoints every 8 hours every day, and applying it gently and evenly.

Effectiveness: the duration of nasal congestion is significantly increased, the number of sneezes is significantly reduced, and the dizziness and headache are significantly relieved.

The results of the validation of the therapeutic effect of aromatic essential oils on rhinitis are shown in Table 2.

TABLE 2
Verification Results of the Therapeutic Effect of
Aromatic Essential Oils on Rhinitis

| | Effective Rate (%) |
|---|---|
| Embodiment 1 | 91 |
| Embodiment 2 | 93 |
| Embodiment 3 | 90 |
| Comparative Embodiment 1 | 79 |
| Comparative Embodiment 2 | 81 |
| Comparative Embodiment 3 | 84 |
| Comparative Embodiment 4 | 76 |
| Comparative Embodiment 5 | 89 |

It can be seen from the data in Table 2 that the aromatic essential oil for clearing nasal passages provided by the invention can effectively treat rhinitis. Compared with the preparation of aromatic essential oil using commercially available raw materials, the aromatic essential oil prepared by the distillation method provided by the invention has a better therapeutic effect on rhinitis; in addition, changing the distillation method of each aromatic essential oil will lead to a decrease in the therapeutic effect of rhinitis to varying degrees.

Finally, it should be noted that the above embodiments are only used to illustrate the technical solutions of the invention rather than to limit it. Although the invention has been described in detail with reference to the above embodiments, ordinary technicians in the relevant field should understand that the specific implementation methods of the invention can still be modified or replaced by equivalents. Any modification or equivalent replacement that does not depart from the spirit and scope of the invention should be covered within the protection scope of the claims in the invention.

1. A method for preparing aromatic essential oil for clearing nasal passages, including the following steps:
using grape seeds, jojoba seeds, Melaleuca alternifolia leaves, Melaleuca viridiflora leaves, Eucalyptus globulus leaves, mint, camphor, and borneol as raw materials, distilling to obtain grape seed oil, jojoba seed oil, Melaleuca alternifolia leaf oil, Melaleuca viridiflora leaf oil, Eucalyptus globulus leaf oil, mint essential oil, camphor oil, and borneol oil, and then mixing the grape seed oil, jojoba seed oil, Melaleuca alternifolia leaf oil, Melaleuca viridiflora leaf oil, Eucalyptus globulus leaf oil, mint essential oil, camphor oil, and borneol oil to obtain the aromatic essential oil for clearing nasal passages.

2. The method for preparing aromatic essential oil for clearing nasal passages of claim 1, wherein the method for preparing the grape seed oil includes the following steps:
grinding grape seeds into a powder through a 60-mesh sieve to obtain grape seed powder, mixing the grape seed powder with water at a solid-to-liquid ratio of 1:(5-10), and distilling at 120° C. for 4-6 hours to obtain the grape seed oil.

3. The method for preparing aromatic essential oil for clearing nasal passages of claim 1, wherein the method for preparing the jojoba seed oil includes the following steps:
grinding jojoba seeds into a powder through a 60-mesh sieve to obtain jojoba seed powder, mixing the jojoba seed powder with water at a solid-to-liquid ratio of 1:(5-10), and distilling at 140° C. for 2-3 hours to obtain the jojoba seed oil.

4. The method for preparing aromatic essential oil for clearing nasal passages of claim 1, wherein the method for preparing the Melaleuca alternifolia leaf oil includes the following steps:
grinding Melaleuca alternifolia leaves into a powder through a 60-mesh sieve to obtain Melaleuca alternifolia leaf powder, mixing the Melaleuca alternifolia leaf powder with hexane-water (volume ratio 3:2) at a solid-to-liquid ratio of 1:(10-15), and distilling at 80° C. for 4-6 hours to obtain the Melaleuca alternifolia leaf oil.

5. The method for preparing aromatic essential oil for clearing nasal passages of claim 1, wherein the method for preparing the Melaleuca viridiflora leaf oil includes the following steps:
grinding Melaleuca viridiflora leaves into a powder through a 60-mesh sieve to obtain Melaleuca viridiflora leaf powder, mixing the Melaleuca viridiflora leaf powder with hexane-water (volume ratio 3:2) at a solid-to-liquid ratio of 1:(10-15), and distilling at 95° C. for 3-5 hours to obtain the Melaleuca viridiflora leaf oil.

6. The method for preparing aromatic essential oil for clearing nasal passages of claim 1, wherein the method for preparing the Eucalyptus globulus leaf oil includes the following steps:
grinding Eucalyptus globulus leaves into a powder through a 60-mesh sieve to obtain Eucalyptus globulus leaf powder, mixing the Eucalyptus globulus leaf powder with ether-water (volume ratio 1:3) at a solid-to-liquid ratio of 1:(4-8), and distilling at 60° C. for 1-3 hours to obtain the Eucalyptus globulus leaf oil.

7. The method for preparing aromatic essential oil for clearing nasal passages of claim 1, wherein the method for preparing the mint essential oil includes the following steps:
grinding mint into a powder through a 60-mesh sieve to obtain mint powder, mixing the mint powder with water at a solid-to-liquid ratio of 1:(5-10), and distilling at 100° C. for 0.5-1.5 hours to obtain the mint essential oil.

8. The method for preparing aromatic essential oil for clearing nasal passages of claim 1, wherein the method for preparing the camphor oil includes the following steps:
grinding camphor into a powder through a 60-mesh sieve to obtain camphor powder, mixing the camphor powder with water at a solid-to-liquid ratio of 1:(5-10), and distilling at 120° C. for 1-2 hours to obtain the mint camphor oil.

9. The method for preparing aromatic essential oil for clearing nasal passages of claim 1, wherein the method for preparing the borneol oil includes the following steps:
grinding borneol into a powder through a 60-mesh sieve to obtain borneol powder, mixing the borneol powder with ether-water (volume ratio 1:3) at a solid-to-liquid ratio of 1:(4-8), and distilling at 50° C. for 2-3 hours to obtain the borneol oil.
